(19) United States
(12) Patent Application Publication
(10) Pub. No.: US 2006/0036274 A1
(43) Pub. Date: Feb. 16, 2006

(54) One-piece vessel harvester

(76) Inventors: Raymond W. Usher, Coon Rapids, MN (US); Cynthia T. Clague, Brooklyn Park, MN (US)

Correspondence Address: Jeffrey J. Hohenshell, 7601 Northland Drive, Brooklyn Park, MN, 55428, US

(21) Appl. No.: 11/147,618

(22) Filed: Jun. 8, 2005

Related U.S. Application Data
(60) Provisional application No. 60/583,163, filed on Jun. 25, 2004.

(51) Int. Cl. A61B 17/00

(52) U.S. Cl. 606/190

(57) ABSTRACT

The invention provides a system and method for harvesting a vessel section. The system comprises an endoscope, a harvesting tool, and an elongated instrument including a transparent distal nose portion, an intermediate portion having a recessed working area, and a proximal portion having at least one passageway to allow the harvesting tool access to and the endoscope visualization of the working area. The method includes making an incision at a point corresponding to the proximal end of the vessel section being harvested. The endoscope is positioned in the nose portion of the instrument, and instrument is inserted through the incision and partially into the body. Tissue is bluntly dissected away from the vessel using the nose portion. The endoscope is repositioned adjacent to the recessed working area, and a harvesting tool is inserted into the working area through the passageway and visually monitored with the endoscope.

This application claims the benefit of U.S. Patent Provisional Application No. 60/583,163 filed Jun. 25, 2004 (the entire contents is incorporated herein by reference).

### TECHNICAL FIELD

This invention relates generally to biomedical systems and methods. More specifically, the invention relates to systems and methods for harvesting a vessel section.

### BACKGROUND OF THE INVENTION

Heart disease, specifically coronary artery disease, is a major cause of death, disability, and healthcare expense in the United States and other industrialized countries. A common form of heart disease is atherosclerosis, in which the vessels leading to the heart are damaged or obstructed by plaques containing cholesterol, lipoid material, lipophages, and other materials. When severely damaged or obstructed, one or more of the vessels can be replaced during a coronary artery bypass graft (CABG) procedure. CABG surgery is performed about 350,000 times annually in the United States, making it one of the most commonly performed major operations.

To prevent rejection, the graft material is preferably a blood vessel harvested from elsewhere within a patient's body. The most commonly used bypass vessel is the saphenous vein from the leg. Because the venous system of the leg is redundant, other veins that remain within the patient's leg are adequate to provide return blood flow following removal of the saphenous vein.

Various methods have been used to harvest the saphenous vein. Until recently, the typical procedure involved making a single long incision, extending from a patient's groin to at least the knee and often to the ankle. This method results in substantial postoperative pain, with patients frequently complaining more about discomfort from the site of leg vein harvesting than about their CABG surgery wound. In addition, the extensive incision site is subject to infection and delayed healing, especially in patients with poor circulation, which not infrequently accompanies coronary artery disease. The disfiguring scar from such a large incision is also of concern to some patients.

Less invasive procedures are preferred, and surgical devices and techniques now exist that allow the saphenous vein to be harvested through one or more small, transverse incisions, generally using an endoscope. Endoscopic procedures yield reduced wound complications, reduced discomfort, and superior cosmetic results compared with traditional methods of vein harvesting. Equipment for endoscopic vein harvesting is frequently complex, requiring multiple devices or requiring that devices and tools be altered or exchanged during the endoscopic procedure. Using such equipment can be time consuming and can require that more than one person be actively involved in carrying out the procedure.

Therefore, it would be desirable to have a system and a method for harvesting a vessel section that overcome the aforementioned and other disadvantages.

### SUMMARY OF THE INVENTION

One aspect of the present invention is a device for harvesting a vessel section, e.g., a saphaneous vein section. The device comprises a distal nose portion, an intermediate portion, and a proximal portion. The intermediate portion has a recessed working area. The proximal portion has at least one passageway to the working area to allow a harvesting tool access to and an endoscope visualization of the working area.

Another aspect of the present invention is a system for harvesting a vessel section, e.g., a saphaneous vein section. The system comprises an endoscope, a harvesting tool, and an elongated instrument including a distal nose portion, an intermediate portion having a recessed working area, and a proximal portion. The proximal portion has at least one passageway to the working area to allow a harvesting tool access to and an endoscope visualization of the working area.

Yet another aspect of the present invention is a method for harvesting a vessel section, e.g., a saphaneous vein section. An incision is made in a body at a point corresponding to a proximal end of the vessel section to be harvested. An elongated instrument including an intermediate portion having a recessed working area is inserted through the incision and partially into the body. A harvesting tool is inserted into the recessed working area through a passageway of the instrument. The harvesting tool is visually monitored with an endoscope.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is an illustration of one embodiment of a device for harvesting a vessel section, in accordance with the present invention;

FIGS. 2A-2E illustrate cross-sectional views of the device of FIG. 1;

FIG. 3 is an illustration of another embodiment of a device for harvesting a vessel section, in accordance with the present invention;

FIGS. 4A-4D illustrate cross-sectional views of the device of FIG. 3;

FIG. 5 is an illustration of one embodiment of a system for harvesting a vessel section, in accordance with the present invention; and

FIG. 6 is a flow diagram of one embodiment of a method for harvesting a vessel section, in accordance with the present invention.

### DETAILED DESCRIPTION OF THE INVENTION

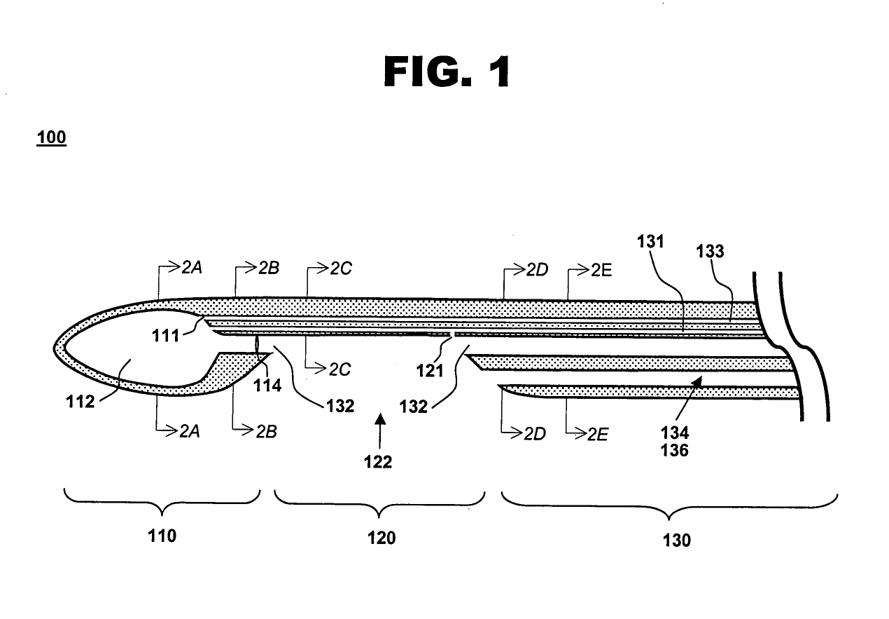

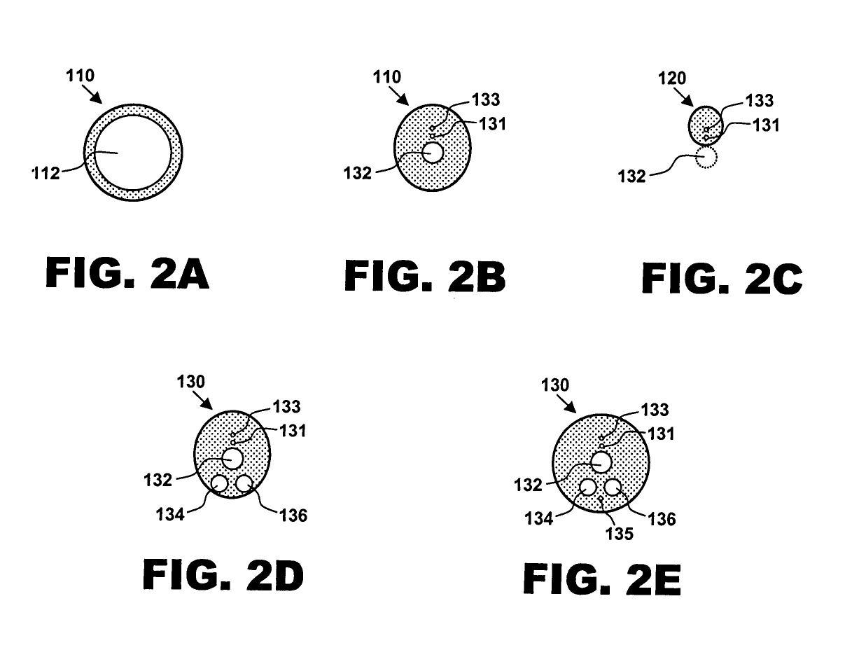

One aspect of the present invention is a device for harvesting a vessel section, e.g., a saphaneous vein section. One embodiment of the device, in accordance with the present invention, is illustrated in FIG. 1 at 100. Device 100 includes a distal nose portion 110, an intermediate portion 120, and a proximal portion 130. As used in this specification, “distal” and “proximal” are with reference to the operator when the device is in use. The three portions form a single, integral device. Cross-sectional views of device 100 are shown in FIGS. 2A-2E. Like elements share like numbers in FIG. 1 and FIGS. 2A-2E.

Distal nose portion 110 is used to bluntly dissect tissue away from the vessel being harvested. Nose portion 110 is made of an appropriate biocompatible material such as a transparent polymer that permits visualization through the nose portion using an endoscope. The transparent material is shaded in FIG. 1 to distinguish it from open areas within the instrument. Nose portion 110 includes a cavity 112, seen in FIG. 1 and FIG. 2A, making the nose portion largely hollow. The nose portion walls have a uniform thickness at the distal end of the device to minimize visual distortion. As illustrated in FIG. 1, nose portion 110 may be roughly cone shaped with a spherical tip, a shape that is efficient for dissecting tissue away from the vessel being harvested. However, the nose portion 110 may assume alternative shapes that are also acceptable for blunt dissection. For example, a roughly spherical nose portion may offer a wider view of the area surrounding the nose portion when an endoscope is positioned within the sphere.

Intermediate portion 120 extends from distal nose portion 110 and comprises the same material as that used to form the nose portion or a different material, such as an alternative plastic and/or metal, e.g., stainless steel. Intermediate portion 120 has a recessed working area 122 within which harvesting tools can be operated to cut and cauterize side branches, for example, or to lift and move the vessel being harvested or the side branches as needed.

Proximal portion 130 extends from intermediate portion 120 and comprises one or more materials that are the same as or different from that used to form the nose portion and the intermediate portion. As illustrated in FIG. 1, proximal portion 130 includes an endoscope passageway 132 and first and second harvesting tool passageways 134 and 136.

Endoscope passageway 132 extends through intermediate portion 120 and into distal nose portion 110 to allow visualization of an area adjacent to distal nose portion 110 in addition to working area 122. Thus, an endoscope may be positioned within nose portion 110 for visualization of the blunt dissection process and then repositioned adjacent to working area 122 to visually monitor the process of cutting and cauterizing side branches, for example.

FIG. 2B shows a cross-sectional view of endoscope passageway 132 entering nose portion 110. FIG. 2C shows dotted lines indicating endoscope passageway 132 adjacent to the solid structure of intermediate portion 120. In the present embodiment, endoscope passageway 132 includes an opening into working area 122, and the endoscope passes outside the device as it is extended through intermediate portion 120, and reenters the device for positioning within cavity 112 of distal nose portion 110. An optional sealing member, is shown in FIG. 1 at 114. The sealing member helps to prevent bodily fluids from entering cavity 112 when the endoscope is positioned for visualization of working area 122. A second sealing member (not shown) might be positioned within endoscope passageway 132 in proximal portion 130; however, the presence of the endoscope within the passageway helps prevent bodily fluids from entering this section of the passageway.

Tool passageways 134 and 136 (behind and parallel to passageway 134) are shown in FIG. 1 and also in cross section in FIGS. 2D and 2E. These passageways allow harvesting tools access to working area 122. Straight passageways are shown in FIG. 1, but the passageways may also be curved and accommodate flexible tools.

Proximal portion 130 includes at least one lumen in fluid communication with a pressurized fluid supply. The fluid may be, for example, a saline solution for rinsing or a gas such as CO₂ for insufflation. As shown in FIGS. 1, 2D, and 2E, lumen 131 is positioned to carry a saline solution to rinse bodily fluids or tissue from the viewing portion of an endoscope received within endoscope passageway 132. The endoscope may become fouled as it passes through working area 122 to alternately view blunt dissection being carried out using nose portion 110 and monitor harvesting tools being used in working area 122. The saline solution can also be used to irrigate working area 122 if desired.

In the present embodiment, lumen 131 extends not only through proximal portion 130, but also through intermediate portion 120 and into distal nose portion 110. Lumen 131 has an intermediate outlet 121 into working area 122 and a distal outlet 111 into cavity 112 of nose portion 110.

When an endoscope is positioned for viewing into working area 122, i.e., the distal end of the endoscope is positioned proximal to outlet 121, saline solution is allowed to exit through outlet 121 to rinse the viewing portion of the endoscope and/or to irrigate working area 122. When the endoscope is positioned in cavity 112 for viewing an area adjacent to nose portion 110, a portion of the endoscope blocks outlet 121, and saline solution is directed to outlet 111 to wash the endoscope at that location.

The saline solution may be allowed to remain in cavity 112 after cleaning the endoscope, with the endoscope viewing through the saline solution as well as the transparent walls of nose portion 110. The saline solution may, in fact, enhance visibility through the nose portion walls. In the present embodiment, a lumen 133 is in communication with suction means (e.g., a vacuum system) to withdraw some or all of the saline solution once cavity 112 becomes filled or if the saline solution becomes fouled, resulting in reduced visibility. Lumen 133 extends through proximal portion 130 and intermediate portion 120 and into cavity 112 in distal nose portion 110. Lumen 133 is shown in cross section in FIGS. 2D and 2E. FIG. 2E also shows an optional lumen 135 in fluid communication with a gas such as CO₂ for insufflation of working area 122. In an alternative embodiment, lumen 133 may be eliminated, and the endoscope may be withdrawn from the nose portion to allow some or all of the saline solution to drain out through endoscope passageway 132 into working area 122.

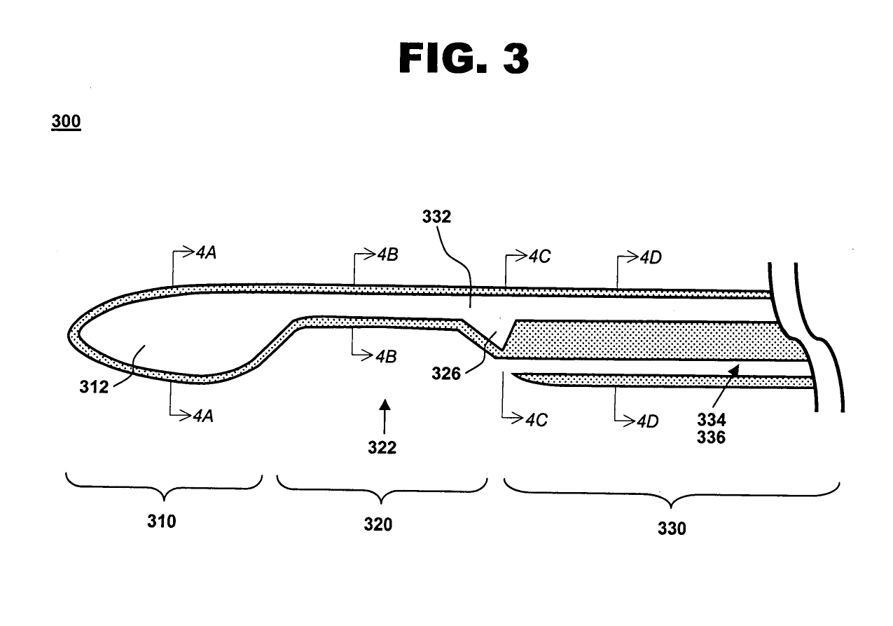

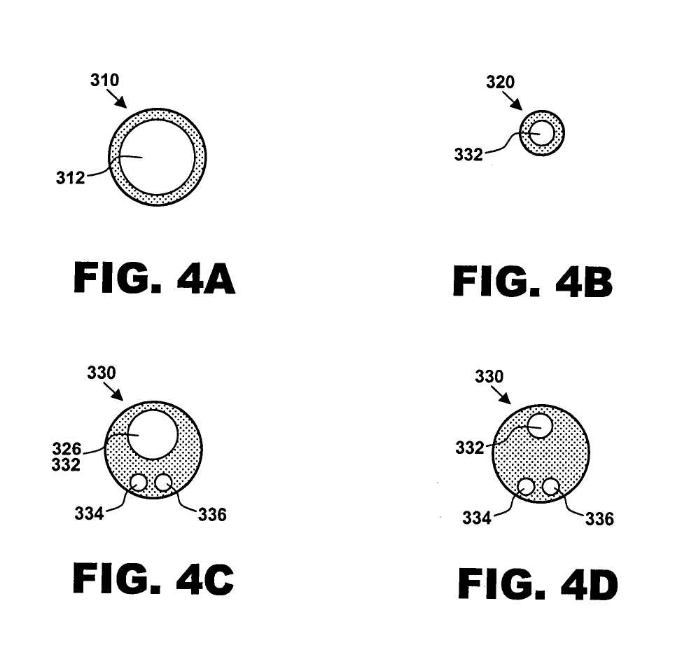

Another embodiment of the device, in accordance with the present Invention, is illustrated in FIG. 3 at 300. Device 300 comprises a distal nose portion 310, an intermediate portion 320, and a proximal portion 330. The three portions form a single, integral device. Cross-sectional views of device 300 are shown in FIGS. 4A-4D. Like elements share like numbers in FIG. 3 and FIGS. 4A-4D.

In the present embodiment, nose portion 310 is similar to the transparent nose portion of device 100, described above. The transparent material is shaded in FIG. 3 to distinguish it from open areas within the instrument. Nose portion 310 includes a cavity 312 that is shown in FIG. 3 and in a cross-sectional view in FIG. 4A. One skilled in the art will appreciate that nose portion 310 may assume other shapes.

Intermediate portion 320 extends from distal nose portion 310, and proximal portion 330 extends from intermediate portion 320. Both portions preferably comprise the same transparent material that was used to form nose portion 310. Intermediate portion 320 has a recessed working area 322 within which harvesting tools can be operated to cut and cauterize side branches, for example, or to lift and move the vessel being harvested or side branches to the vessel.

As seen in FIGS. 3 and 4B, an endoscope passageway 332 extends through all three portions of the device. In this embodiment, an endoscope remains sealed within the passageway and is not exposed to bodily fluids or tissues at any time. The endoscope may be positioned within nose portion 310 to view blunt dissection or adjacent to a viewing area 326 that extends out from passageway 332 to monitor harvesting tools being used in working area 322. Harvesting tools gain access to working area 322 through harvesting tool passageways 334 and 336, shown in FIGS. 3, 4C, and 4D. Straight tool passageways are shown in FIG. 3, but the passageways may also be curved and accommodate flexible tools.

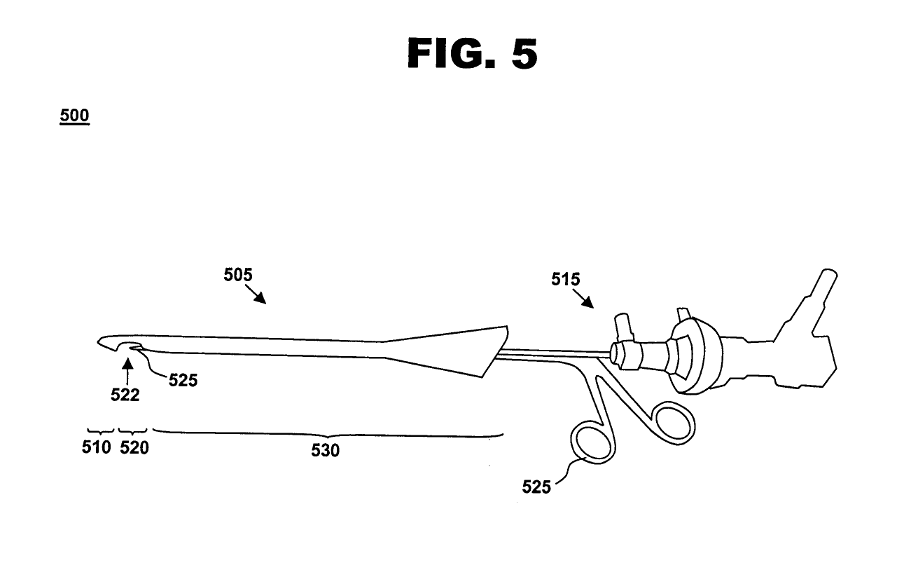

Another aspect of the present invention is a system for harvesting a vessel section. One embodiment of the system, in accordance with the present invention, is illustrated in FIG. 5 at 500. System 500 comprises an elongated instrument 505, an endoscope 515, and at least one harvesting tool 525.

Elongated instrument 505 may be a device such as has been described above and illustrated in FIGS. 1-4D. In the present embodiment, elongated instrument 505 is similar to device 100, illustrated in FIGS. 1 and 2A-2E. Elongated instrument 505 includes a distal nose portion 510, an intermediate portion 520, and a proximal portion 530. The three portions form a single, integral device.

Distal nose portion 510 is used to bluntly dissect tissue away from the vessel being harvested. In the present embodiment, nose portion 510 is made of a transparent polymer that permits visualization of the blunt dissection process through the walls of nose portion 510 using endoscope 515. Nose portion 510 includes a cavity such as that shown in FIG. 1 at 112, making the nose portion largely hollow with walls having a uniform thickness to minimize visual distortion. As illustrated in FIG. 5, nose portion 510 is roughly cone shaped with a spherical tip. However, other shapes, for example a roughly spherical shape, are possible.

Intermediate portion 520 extends from distal nose portion 510, and proximal portion 530 extends from intermediate portion 520. In the present embodiment, intermediate portion 520 and proximal portion 530 comprise the same transparent polymer that was used to form nose portion 510.

Intermediate portion 520 has a recessed working area 522 within which harvesting tools are operated. A cutting and cauterizing tool 525 is shown in FIG. 5. Additional tools may be included in the system. For example, system 500 may include a tool to lift and move the vessel being harvested or side branches of the vessel. Proximal portion 530 includes at least one tool passageway such as those shown at 134 and 136 in FIGS. 1, 2D, and 2E to allow the harvesting tool(s) access to working area 522. A tool passageway may be straight to accommodate a rigid tool or curved where a flexible tool is employed. In the present embodiment, each tool remains in place within its tool passageway and is an integral part of system 500. This design eliminates the inconvenience of having to insert tools as they are needed or having to withdraw a tool to make way for another tool that is needed. One skilled in the art will appreciate that the tools may be removable in another embodiment.

An endoscope passageway such as that shown in FIG. 1 at 132 extends through proximal portion 530 and intermediate portion 530 and into distal nose portion 510. Endoscope 515 is movable within the endoscope passageway and may be positioned within nose portion 510 for visualization of the blunt dissection process and then repositioned adjacent to working area 522 to visually monitor harvesting tools operating in that area. Endoscope 515 is, preferably, a four- or 5-millimeter (4- or 5-mm) thirty-degree (30°) endoscope; however, other endoscopes may be used in the present invention.

The endoscope passageway includes an opening into working area 522 such as is shown in FIG. 1. Endoscope 515 passes outside the device as it is extended through intermediate portion 520, and reenters the device for positioning within distal nose portion 510. A sealing member (not shown) may be included within the endoscope passageway to help prevent bodily fluids from entering the cavity within nose portion 510 when the endoscope is positioned for visualization of working area 522.

Proximal portion 530 includes a lumen in fluid communication with a pressurized fluid supply such as the lumen shown in FIG. 1 at 131. The fluid may be, for example, a saline solution for rinsing or a gas such as CO₂ for insufflation. In the present embodiment, the lumen is intended to carry a saline solution to be used as needed for rinsing bodily fluids or tissue from the viewing portion of endoscope 515. The endoscope may become fouled as it passes through working area 522 to alternately view blunt dissection being carried out using nose portion 510 and monitor tools being used in working area 522. The saline solution can also be used to irrigate working area 522 if desired.

In the present embodiment, the saline solution lumen extends through proximal portion 530 and intermediate portion 520 and into nose portion 510. The lumen has an intermediate outlet into working area 522 and a distal outlet into the cavity of nose portion 510. When endoscope 515 is positioned for viewing into working area 522, i.e., the distal end of the endoscope is positioned proximal to the intermediate outlet, saline solution is allowed to exit through the intermediate outlet. When endoscope 515 is positioned within nose portion 510, a portion of the endoscope blocks the intermediate outlet, and saline solution is directed to the distal outlet to wash the endoscope at that location.

The saline solution may be allowed to remain in the cavity of distal nose portion 510 after cleaning the endoscope, with the endoscope viewing through the saline solution as well as the transparent walls of nose portion 510. The saline solution may, in fact, enhance visibility through the nose portion walls. In the present embodiment, a second lumen such as that shown at 133 in FIG. 1 is provided to withdraw some or all of the saline solution once the cavity of distal nose portion 510 becomes filled or if the saline solution becomes fouled, resulting in reduced visibility. This second lumen is attached to suction means (e.g., a vacuum system) adjacent to the proximal end of the device and extends through proximal portion 530 and intermediate portion 520 and into the cavity of distal nose portion 510.

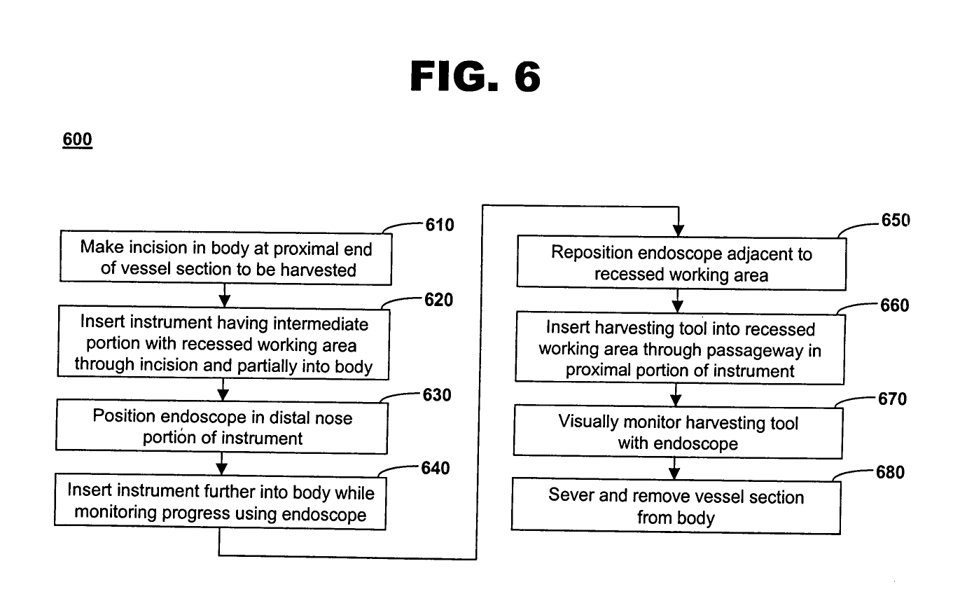

Yet another aspect of the present invention is a method for harvesting a vessel section, e.g., a saphaneous vein section. FIG. 6 shows a flow diagram of one embodiment of the method in accordance with the present invention.

An incision is made in a body at a point corresponding to a proximal end of the vessel section to be harvested (Block 610). An elongated instrument with an intermediate portion having a recessed working area is inserted through the incision and partially into the body (Block 620). In the present embodiment, the elongated instrument is a device such as those described above, having a transparent distal nose portion that is used to bluntly dissect tissue away from the vessel being harvested and a proximal portion that includes at least one passageway to allow a harvesting tool access to the working area.

An endoscope is positioned in the distal nose portion of the elongated instrument (Block 630), and the instrument is inserted further into the body while monitoring the progress of the instrument using the endoscope (Block 640). As described above with regard to devices 100 and 300 and system 500, the elongated instrument includes an endoscope passageway that allows the endoscope to be positioned both within the distal nose portion and adjacent to the recessed working area.

The endoscope is positioned initially within the distal nose portion for viewing the blunt dissection process. The endoscope is then repositioned adjacent to the recessed working area (Block 650), and a harvesting tool is inserted into the working area through a passageway in the proximal portion of the instrument (Block 660). The harvesting tool is visually monitored with the endoscope while it, for example, cuts and cauterizes side branches (Block 670).

The steps of Blocks 630 through 670 may be repeated until the vessel section to be harvested is free of adjoining tissue and side branches. Thus, the endoscope is moved back and forth between the nose portion and the working area as the instrument is advanced into the body, first bluntly dissecting tissue away from a section of the vessel using the nose portion, and then cutting the vessel free using harvesting tools within the working area. The vessel section is then severed and removed from the body (Block 680).

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

What is claimed is:

1. A device for harvesting a vessel section, comprising:
a distal nose portion;
an intermediate portion having a recessed working area; and
a proximal portion having at least one passageway to the working area to allow a harvesting tool access to and an endoscope visualization of the working area.

2. The device of claim 1 wherein the at least one passageway comprises a first passageway to receive an endoscope and a second passageway to receive a harvesting tool.

3. The device of claim 2 wherein the first passageway further extends through the intermediate portion and into the distal nose portion to allow an endoscope visualization of an area adjacent to the distal nose portion.

4. The device of claim 3 wherein the first passageway includes an opening into the working area.

5. The device of claim 4 wherein the first passageway includes at least one sealing member adjacent to the opening into the working area.

6. The device of claim 2 wherein the second passageway is one of straight or curved.

7. The device of claim 2 wherein the at least one passageway further comprises a third passageway to receive a second harvesting tool.

8. The device of claim 1 wherein the proximal portion includes at least one lumen in communication with a pressurized fluid supply, the lumen having an outlet into the working area.

9. The device of claim 8 wherein the lumen further extends through the intermediate portion and into the distal nose portion, the lumen having a second outlet into a cavity within the distal nose portion.

10. The device of claim 9 wherein the outlet into the working area is closed when a portion of an endoscope is positioned distal to the outlet into the working area.

11. The device of claim 9 wherein a second lumen extends through the proximal portion and the intermediate portion and into the cavity within the distal nose portion, the second lumen in communication with suction means.

12. The device of claim 1 wherein the distal nose portion is substantially spherical.

13. The device of claim 1 wherein the distal nose portion is substantially cone shaped with a spherical distal tip.

14. The device of claim 1 wherein the distal nose portion is substantially hollow.

15. The device of claim 1 wherein at least the distal nose portion is transparent.

16. A system for harvesting a vessel section, comprising:
an endoscope;
a harvesting tool; and
an elongated instrument including a distal nose portion, an intermediate portion having a recessed working area, and a proximal portion having at least one passageway to the working area to allow the harvesting tool access to and the endoscope visualization of the working area;

17. The system of claim 16 wherein the at least one passageway comprises a first passageway to receive the endoscope and a second passageway to receive the harvesting tool.

18. The system of claim 17 wherein the first passageway further extends through the intermediate portion and into the distal nose portion to allow the endoscope visualization of an area adjacent to the distal nose portion.

19. The system of claim 18 wherein the first passageway includes an opening into the working area.

20. The system of claim 19 wherein the first passageway includes at least one sealing member adjacent to the opening into the working area.

21. The system of claim 17 wherein the second passageway is straight.

22. The system of claim 17 wherein the second passageway is curved.

23. The system of claim 22 wherein the harvesting tool is flexible.

24. The system of claim 17 wherein the at least one passageway further comprises a third passageway to receive a second harvesting tool.

25. The system of claim 16 wherein the proximal portion includes at least one lumen in communication with a pressurized fluid supply, the lumen having an outlet into the working area.

26. The system of claim 25 wherein the lumen further extends through the intermediate portion and into the distal nose portion, the lumen having a second outlet into a cavity within the distal nose portion.

27. The system of claim 26 wherein the outlet into the working area is closed when a portion of an endoscope is positioned distal to the outlet into the working area.

28. The system of claim 26 wherein the elongated instrument includes at least one lumen in communication with suction means, the lumen having an outlet into the cavity within the distal nose portion.

29. The system of claim 16 wherein the distal nose portion is substantially spherical.

30. The system of claim 16 wherein the distal nose portion is substantially cone-shaped with a spherical distal tip.

31. The system of claim 16 wherein the distal nose portion is substantially hollow.

32. The system of claim 16 wherein at least the distal nose portion is transparent.

33. A method for harvesting a vessel section, the method comprising:
making an incision in a body at a point corresponding to a proximal end of the vessel section to be harvested;
inserting an elongated instrument through the incision and partially into the body, the elongated instrument including an intermediate portion having a recessed working area;
inserting a harvesting tool into the recessed working area through a passageway of the instrument; and
visually monitoring the harvesting tool with an endoscope.

34. The method of claim 33 further comprising:
positioning an endoscope in a distal nose portion of the elongated instrument; and
inserting the instrument further into the body while visually monitoring the progress of the instrument using the endoscope.

35. The method of claim 33 further comprising:
repositioning the endoscope to a position adjacent to the recessed working area while operating the harvesting tool.
